(19) United States
(12) Patent Application Publication
(10) Pub. No.: US 2016/0146769 A1
(43) Pub. Date: May 26, 2016

(54) METHODS AND DEVICES FOR ACQUIRING AIR QUALITY

(71) Applicant: Xiamomi Inc., Beijing (CN)

(72) Inventors: Bin Zhang, Beijing (CN); Jun Su, Beijing (CN); Yongfeng Xia, Beijing (CN); Yunlin Hu, Beijing (CN); Yang Wang, Beijing (CN); Hao Chen, Beijing (CN)

(73) Assignee: Xiaomi Inc.

(21) Appl. No.: 14/832,752

(22) Filed: Aug. 21, 2015

Related U.S. Application Data
(63) Continuation of application No. PCT/CN2015/077823, filed on Apr. 29, 2015.

(30) Foreign Application Priority Data
Nov. 21, 2014 (CN) 201410675672.2

(51) Int. Cl. G01N 33/00

(52) U.S. Cl. CPC G01N 33/0063; G01N 33/0073

(57) ABSTRACT

A method for a device to acquire air quality, includes: controlling a fan to rotate; measuring air quality by an air-quality detector; and generating air-quality information according to a measurement result of the air-quality detector.

### CROSS-REFERENCE TO RELATED APPLICATIONS

The present application is a continuation of International Application No. PCT/CN2015/077823, filed Apr. 29, 2015, which is based upon and claims priority to Chinese Patent Application No. 201410675672.2, filed Nov. 21, 2014, the entire contents of all of which are incorporated herein by reference.

### TECHNICAL FIELD

The present disclosure generally relates to the field of monitoring technology and, more particularly, to methods and devices for acquiring air quality.

### BACKGROUND

Air purifiers are used to improve air quality. Conventionally, a typical air purifier carries an air-quality detector to measure the air quality in an environment surrounding the air purifier. However, when the ventilation is poor, the air-quality detector may not accurately measure a concentration of dust particles suspending in the air. The air quality measured in this manner may be higher than the actual air quality.

### SUMMARY

According to a first aspect of the present disclosure, there is provide a method for a device to acquire air quality, comprising: controlling a fan to rotate; measuring air quality by an air-quality detector; and generating air-quality information according to a measurement result of the air-quality detector.

According to a second aspect of the present disclosure, there is provide a device for acquiring air quality, comprising: a processor; and a memory for storing instructions executable by the processor; wherein the processor is configured to perform: controlling a fan to rotate; controlling an air-quality detector to measure air quality; and generating air-quality information according to a measurement result of the air quality detector.

According to a third aspect of the present disclosure, there is provide a non-transitory computer-readable storage medium storing instructions that, when executed by a processor of a device, cause the device to perform a method for acquiring air quality, the method comprising: controlling a fan to rotate; measuring air quality by an air-quality detector; and generating air-quality information according to a measurement result of the air quality detector.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments consistent with the disclosure and, together with the description, serve to explain the principles of the present disclosure.

FIG. 1 is a schematic diagram illustrating an implementation environment of a method for acquiring air quality, according to an exemplary embodiment.

FIG. 2 is a flowchart of a method for acquiring air quality, according to an exemplary embodiment.

FIG. 3A is a flowchart of a method for acquiring air quality, according to an exemplary embodiment.

FIG. 3B is a schematic diagram illustrating a mobile-terminal interface for sending a trigger signal for triggering measurement of air quality, according to an exemplary embodiment.

FIG. 3C is a schematic diagram illustrating a mobile-terminal interface for displaying air-quality information, according to an exemplary embodiment.

FIG. 3D is a schematic diagram illustrating a mobile-terminal interface for displaying a progress of measuring air quality, according to an exemplary embodiment.

FIG. 4 is a flowchart of a method for acquiring air quality, according to an exemplary embodiment.

FIG. 5 is a block diagram of a device for acquiring air quality, according to an exemplary embodiment.

FIG. 6 is a block diagram of a device for acquiring air quality, according to an exemplary embodiment.

FIG. 7 is a block diagram of a device for acquiring air quality, according to an exemplary embodiment.

### DESCRIPTION OF THE EMBODIMENTS

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the present disclosure. Instead, they are merely examples of devices and methods consistent with aspects related to the invention as recited in the appended claims.

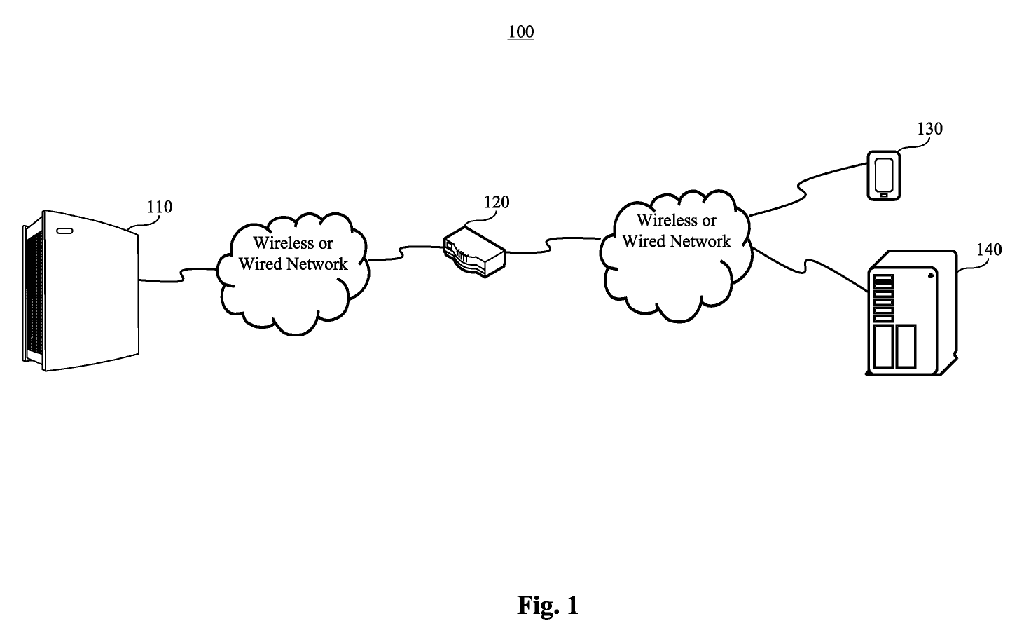

FIG. 1 is a schematic diagram illustrating an implementation environment 100 of a method for acquiring air quality, according to an exemplary embodiment. Referring to FIG. 1, the implementation environment 100 may include an appliance 110, a gateway device 120, a mobile terminal 130, and a server 140.

The appliance 110 may include a fan and an air-quality detector. For example, the appliance 110 may be an air purifier. The fan is configured to drive air flow in an environment surrounding the appliance. The air-quality detector is configured to detect the air quality in the surrounding environment. The fan may be a fan originally built in the appliance 110, or a later-added fan placed near the air-quality detector. The appliance 110 may connect to the gateway device 120 through a wired or wireless network, and further connect to the mobile terminal 130 and the server 140 through the gateway device 120.

The mobile terminal 130 may be installed with an application provided by a service provider. A user may control the appliance 110 through the application. When the mobile terminal 130 and the appliance 110 connect to the same local area network (LAN), the mobile terminal 130 may search for the appliance 110 through the gateway device 120, and establish a binding relationship with the appliance 110. The appliance 110 may maintain binding relationships with more than one mobile terminal 130.

The server 140 may be a back-end server managed by the service provider. The server 140 may work with the mobile terminal 130 to perform services provided by the service provider.

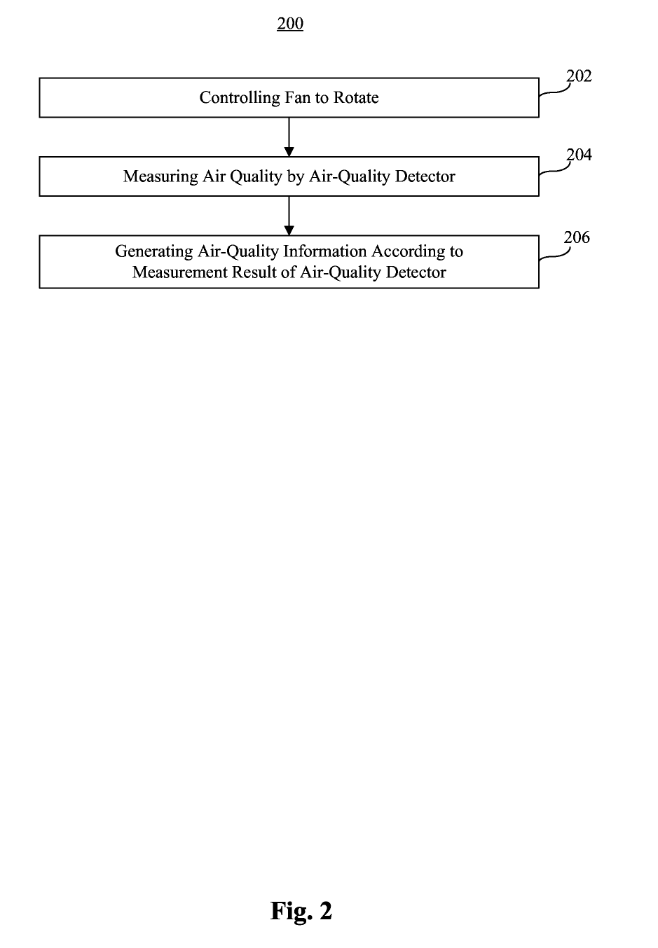

FIG. 2 is a flowchart of a method 200 for acquiring air quality, according to an exemplary embodiment. For example, the method 200 may be applied in the appliance 110 (FIG. 1). Referring to FIG. 2, the method 200 may include the following steps.

In step 202, the appliance controls a fan to rotate.

In step 204, when the air surrounding the appliance flows, the appliance measures air quality by an air-quality detector.

In step 206, the appliance generates air-quality information according to a measurement result of the air-quality detector.

Because the method 200 measures the air quality when the surrounding air is flowing, a concentration of dust particles in the air can be measured accurately.

In exemplary embodiments, the appliance may start measuring the air-quality in the following two situations.

In the first situation, the appliance measures the air quality after receiving a trigger signal from a mobile terminal The trigger signal is configured to trigger the measurement of air quality.

In the second situation, the appliance measures the air quality when a predetermined time is reached. The predetermined time may be one of a plurality of times at a predetermined time interval, or a time set by a user.

The above two situations are described in detail in the following two exemplary embodiments, respectively.

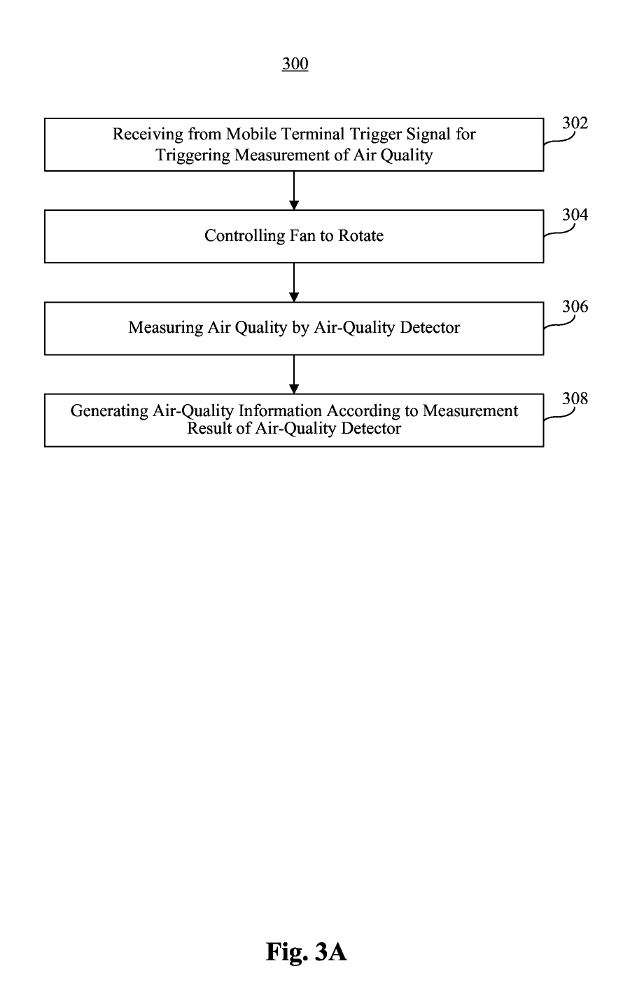

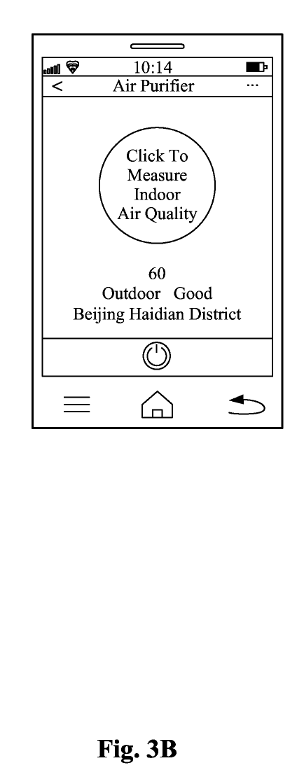

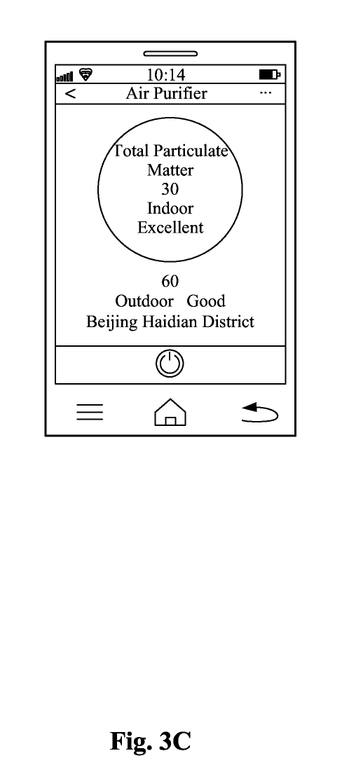

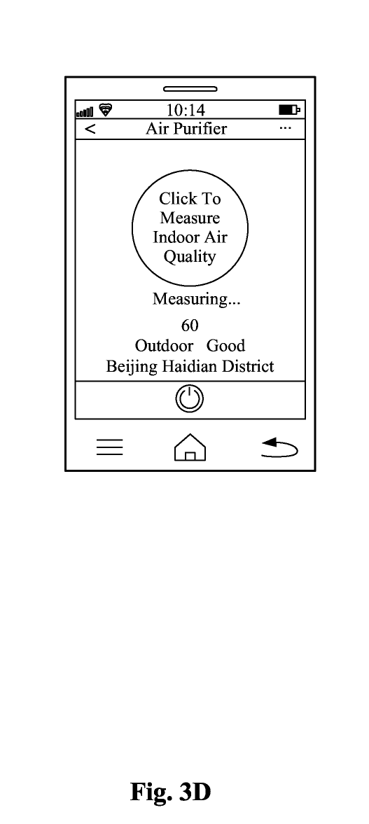

FIG. 3A is a flowchart of a method 300 for acquiring air quality, according to an exemplary embodiment. For example, the method 300 may be applied in the appliance 110 (FIG. 1) and the appliance 110 may be an air purifier including a fan and an air-quality detector. Referring to FIG. 3A, the method 300 may include the following steps.

In step 302, the air purifier receives a trigger signal sent by a mobile terminal. The trigger signal is configured to trigger the measurement of air quality.

For example, when a user wants to know the air quality, the user may operate a mobile terminal, such as the mobile terminal 130 (FIG. 1), to send the air purifier a trigger signal for triggering the measurement of the air quality.

FIG. 3B is a schematic diagram illustrating a mobile-terminal interface for triggering the measurement of air quality, according to an exemplary embodiment. Referring to FIG. 3B, for example, the mobile terminal may be a mobile phone installed with an application for controlling an air purifier at home. When the user wants to know the air quality at home, the user may click the “click to measure indoor air quality” button displayed in the air-purifier application to start an air-quality measurement. After detecting the clicking operation, the mobile phone may send a trigger signal for triggering measurement of the air quality to the air purifier.

In step 304, the air purifier controls the fan to rotate.

The rotation of the fan drives the air surrounding the air purifier to flow. The fan may be a fan originally built in the air purifier, or a later-added fan placed near to the air-quality detector.

In step 306, the air purifier measures the air quality by the air-quality detector.

In step 308, the air purifier generates air-quality information according to a measurement result of the air-quality detector.

In exemplary embodiments, the air-quality information may include at least one of an air-quality level, an air-quality index (AQI), content of fine particles, content of inhalable particles, content of sulfur dioxide, content of nitrogen dioxide, content of ozone, and content of carbonic monoxide.

In exemplary embodiments, the air purifier may return the generated air-quality information to the user in at least one of the following three manners.

In the first manner, the air purifier sends the air-quality information directly to the mobile terminal, such that the mobile terminal displays the air-quality information to the user.

In the second manner, the air purifier sends the air-quality information to a server, such as the server 140 (FIG. 1). The server subsequently sends the air-quality information to the mobile terminal for displaying. For example, the server may be a back-end server belonging to a service producer who provides service to the user through the mobile terminal. The server may send the air-quality information to the mobile terminal according to the account information associated with an air-purifier application.

In the third manner, the air purifier sends the air-quality information to a gateway device, such as the gateway device 120 (FIG. 1). The gateway device subsequently sends the air-quality information to the mobile terminal for displaying.

FIG. 3C is a schematic diagram illustrating a mobile-terminal interface for displaying air-quality information, according to an exemplary embodiment. Referring to FIG. 3C, the mobile terminal may be a mobile phone capable of displaying indoor air-quality information generated by an air purifier. For example, the mobile phone displays that the indoor air-quality level is excellent and the indoor total particulate matter is 30. In addition, the mobile phone may display outdoor air-quality information received from other sources, such as the server of the service provider.

In some embodiments, after the mobile terminal sends the trigger signal to the air purifier but before the mobile terminal receives and displays the air-quality information, the mobile terminal may indicate a real-time progress of measuring the air quality. FIG. 3D is a schematic diagram illustrating a mobile-terminal interface for displaying a progress of measuring air quality, according to an exemplary embodiment. Referring to FIG. 3D, after the user clicks the “click to detect the indoor air quality’ in the air-purifier application (FIG. 3B), the mobile phone displays a message indicating that the air purifier is measuring the air quality.

In some embodiments, the user does not have to trigger the measurement of the air quality through the mobile terminal For example, the air purifier may include a switch for turning on and off the air-quality detector, such that the user may use the switch to trigger the measurement of the air quality.

In some embodiments, the air-quality information may also be directly displayed on the air purifier. For example, the air purifier may include one or more indicator lights for representing the measured air quality. A green light may represent that the air quality is excellent. A yellow light may represent that the air quality is good. And a red light may represent the air is polluted. For another example, the air purifier may include a screen to display the air-quality information to the user.

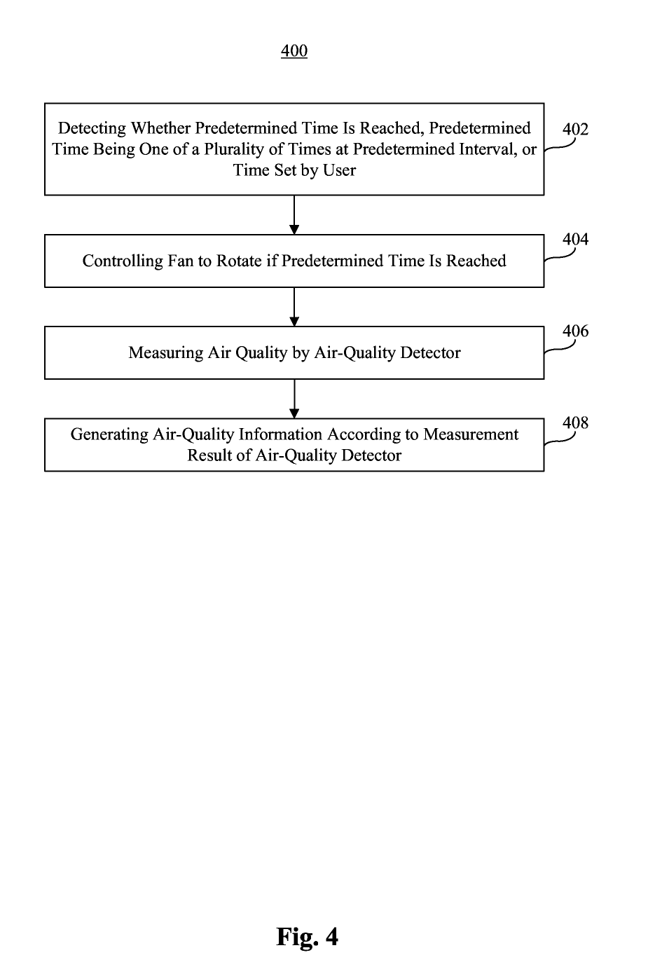

FIG. 4 is a flowchart of a method 400 for acquiring air quality, according to an exemplary embodiment. For example, the method 400 may be applied in the appliance 110 (FIG. 1) and the appliance 110 may be an air purifier capable of measuring air quality. Referring to FIG. 4, the method 400 may include the following steps.

In step 402, the appliance detects whether a predetermined time is reached.

The predetermined time may be one of a plurality of times at a predetermined time interval. The predetermined time interval may be a default time interval of the air purifier, or a time interval set by a user. For example, the user may enter in a mobile phone a command of measuring indoor air quality on the hour. The mobile phone then sends the command to the air purifier. This way, the predetermined time is one o'clock, two o'clock, three o'clock, and the like.

The predetermined time may also be a time set by the user. For example, the user may enter a command of measuring indoor air quality at 5:30 pm. This way, the predetermined time is 5:30 pm.

In step 404, the air purifier controls the fan to rotate if the predetermined time is reached.

The air purifier may start a motor of the fan and rotate the fan through the motor. The rotation of the fan drives the air surrounding the appliance to flow. The fan may be a fan originally built in the air purifier, or a later-added fan placed near to the air-quality detector.

In step 406, the air purifier measures the air quality by the air-quality detector.

The air-quality detector may be a detector originally built in the air purifier. The air purifier may control the air-quality detector to measure the air quality after the surrounding air starts to flow.

In step 408, the air purifier generates air-quality information according to a measurement result of the air-quality detector.

In exemplary embodiments, the generated air-quality information may include at least one of an air-quality level, an AQI, content of fine particles, content of inhalable particles, content of sulfur dioxide, content of nitrogen dioxide, content of ozone, and content of carbonic monoxide.

In exemplary embodiments, the air purifier may return the generated air-quality information to the user in at least one of two manners.

In the first manner, the air purifier sends the air-quality information to a server, such as the server 140 (FIG. 1). When the server receives a trigger signal from a mobile terminal, such as the mobile terminal 130 (FIG. 1), the server sends the air-quality information to the mobile terminal for displaying.

For example, the mobile terminal may send the trigger signal to the server when the user performs a long-press operation on an information-acquisition button of the air-purifier application installed in the mobile terminal After receiving the trigger signal, the server may send all the historically received air-quality information to the mobile terminal. Alternatively, the server may send the most recently received air-quality information to the mobile terminal. In both situations, the mobile terminal displays the received air-quality information.

In exemplary embodiments, the mobile terminal may automatically send the trigger signal to the server when: (1) the user lights a screen of the mobile terminal; (2) the user unlocks a screen of the mobile terminal; (3) the user starts the air-purifier application; or (4) the mobile terminal connects to a network.

In the second manner, the air purifier sends the generated air-quality information to a gateway device, such as the gateway device 120 (FIG. 1). When the gateway device receives a trigger signal from a mobile terminal, the gateway device sends the air-quality information to the mobile terminal for displaying. Similar to the first manner, the gateway device may send the mobile terminal all the historically received air-quality information, or the most recently received air-quality information.

By using the method 400, when the predetermined time is reached, the appliance automatically controls the fan to rotate and controls the air-quality detector to measure the air quality. Thus, the method 400 simplifies user operations needed for acquiring the air quality.

In the methods 200, 300, and 400, the controlling of the fan by the appliance may include the following steps.

First, the appliance detects whether an air-flowing speed in an environment surrounding the appliance reaches a predetermined speed.

Second, the appliance controls the fan to rotate if the air-flowing speed does not reach the predetermined speed. The rotation of the fan can increase the air-flowing speed and thus avoid inaccurate measurement of the air quality caused by, e.g., poor ventilation.

In contrast, if the air-flowing speed reaches the predetermined speed, the appliance may measure the air quality by the air quality detector without rotating the fan.

Moreover, in the methods 200, 300, and 400, the measuring of the air quality by the air-quality detector may include the following steps.

First, the appliance detects whether a rotation time of the fan reaches a predetermined length of time. For example, if the predetermined length of time is 30 seconds, the appliance may detect whether the 30 seconds have elapsed after the fan starts rotation.

Second, the air-quality detector measures the air quality if the rotation time reaches the predetermined length of time. The reaching of the predetermined length of time suggests that the air surrounding the appliance flows at such a speed that the air quality can be accurately measured.

Furthermore, in the methods 200, 300, and 400, the appliance may be an air purifier. When the measurement result of the air quality indicates that the current air quality is poor, the air purifier may automatically start to purify air. Alternatively, the air purifier may remind the user that the air quality is poor and wait for the user's instruction indicating whether to purify the air. For example, the air purifier may generate a warning, such as sounding an alarm, turning on an indicator light, and making a prompt tone, so that the user may be alerted and trigger a purification operation by the air purifier. For another example, the air purifier may send the mobile terminal a prompt message indicating that the current air quality is poor and recommending the user to turn on the air purifier. The mobile terminal is configured to display the prompt message and control the air purifier to purify the air when receiving user instructions.

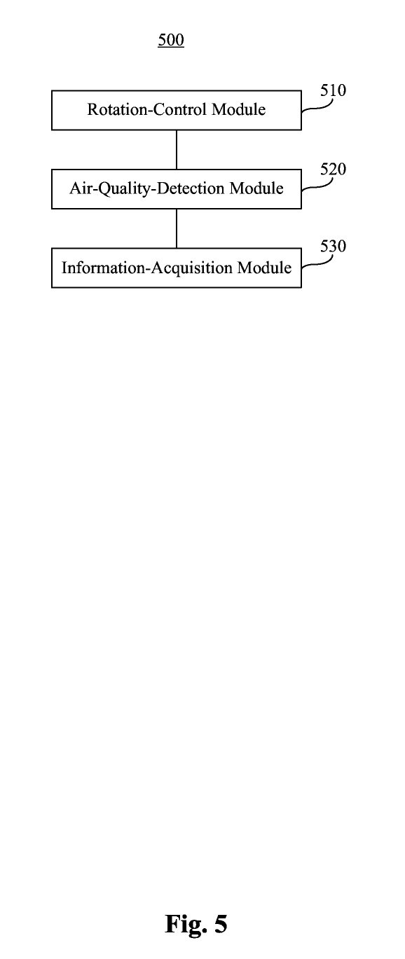

FIG. 5 is a block diagram of a device 500 for acquiring air quality, according to an exemplary embodiment. For example, the device 500 may be the appliance 110 (FIG. 1). Referring to FIG. 5, the device 500 may include a rotation-control module 510, an air-quality-detection module 520, and an information-acquisition module 530.

The rotation-control module 510 is configured to control a fan to rotate.

The air-quality-detection module 520 is configured to measure air quality by an air-quality detector when the air surrounding the appliance is flowing.

The information-acquisition module 530 is configured to generate air-quality information according to a measurement result of the air-quality detector.

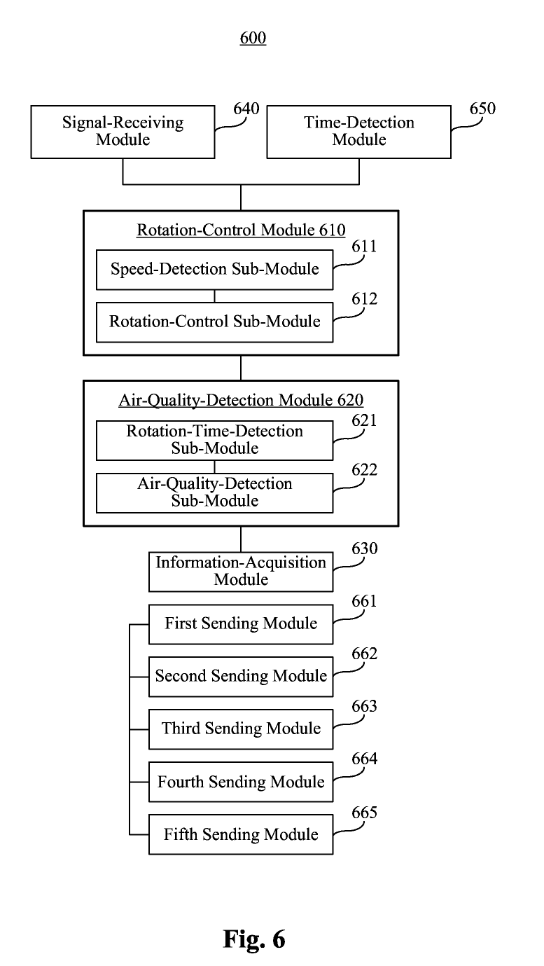

FIG. 6 is a block diagram of a device 600 for acquiring air quality, according to an exemplary embodiment. For example, the device 600 may be the appliance 110 (FIG. 1). Referring to FIG. 1, the device 600 may include a rotation-control module 610, an air-quality-detection module 620, and an information-acquisition module 630, similar to the rotation-control module 510, the air-quality-detection module 520, and the information-acquisition module 530 (FIG. 5), respectively.

In exemplary embodiments, referring to FIG. 6, the rotation-control module 610 may further include a speed-detection sub-module 611 and a rotation-control sub-module 612. The speed-detection sub-module 611 is configured to detect whether the air-flowing speed in an environment surrounding the device 600 reaches a predetermined speed. The rotation-control sub-module 612 is configured to control the fan to rotate if the air-flowing speed does not reach the predetermined speed.

In exemplary embodiments, the air-quality-detection module 620 may further include a rotation-time-detection sub-module 621 and an air-quality-detection sub-module 622. The rotation-time-detection sub-module 621 is configured to detect whether the rotation time of the fan reaches a predetermined length of time. The air-quality-detection sub-module 622 is configured to control an air-quality detector to measure the air quality when the rotation time reaches the predetermined length of time.

In exemplary embodiments, the device 600 may further include a signal-receiving module 640 configured to receive a trigger signal sent by a mobile terminal The trigger signal is configured to trigger the measurement of the air quality, such that the rotation-control module 610 controls the fan to rotate after the signal-receiving module 640 receives the trigger signal.

In exemplary embodiments, the device 600 may further include at least one of a first sending module 661, a second sending module 662, and a third sending module 663. The first sending module 661 is configured to send the air-quality information generated by the information-acquisition module 630 to the mobile terminal for displaying. The second sending module 662 is configured to send the air-quality information generated by the information-acquisition module 630 to a server, such that the server subsequently sends the air-quality information to the mobile terminal for displaying. The third sending module 663 is configured to send the air-quality information generated by the information-acquisition module 630 to a gateway device, such that the gateway device subsequently sends the air-quality information to the mobile terminal for displaying.

In exemplary embodiments, the device 600 may further include a time-detection module 650 configured to detect whether a predetermined time is reached, such that the rotation-control module 610 controls the fan to rotate if the predetermined time is reached. The predetermined time may be one of a plurality of times at a predetermined time interval, or a time set by a user.

In exemplary embodiments, the device 600 may further include at least one of a fourth sending module 664 and a fifth sending module 665. The fourth sending module 664 is configured to send the air-quality information generated by the information-acquisition module 630 to a server. Similarly, the fifth sending module 665 is configured to send the air-quality information generated by the information-acquisition module 630 to a gateway device. Each of the server and the gateway device is figured to send all the historically received air-quality information or the most recently received air-quality information to a mobile terminal, after receiving from the mobile terminal a trigger signal for displaying air-quality information.

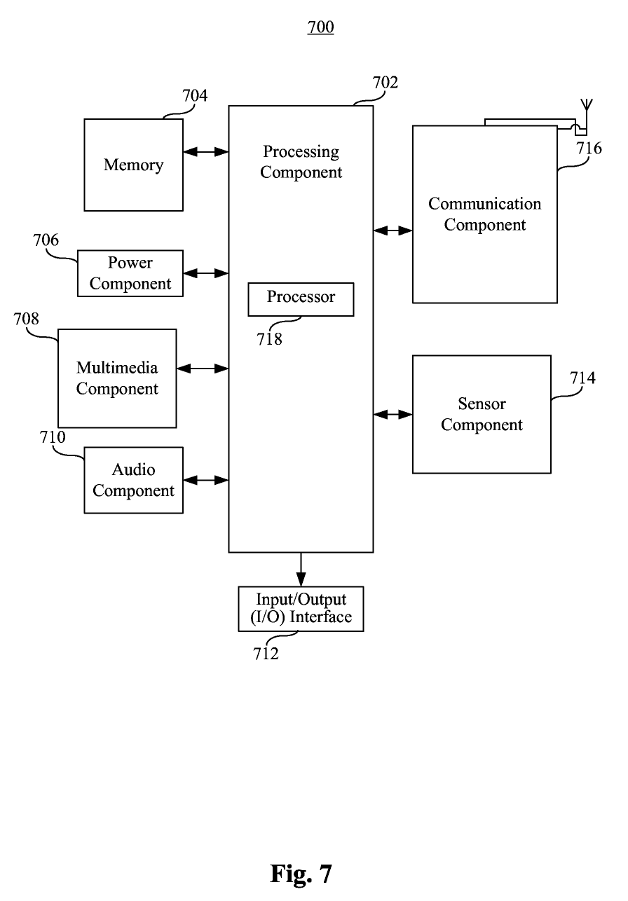

FIG. 7 is a block diagram of a device 700 for acquiring air quality, according to an exemplary embodiment. For example, the device 700 may be an air purifier, a mobile phone, a computer, a gateway device, a digital broadcast terminal, a messaging device, a gaming console, a tablet, a medical device, exercise equipment, a personal digital assistant (PDA), and the like.

Referring to FIG. 7, the device 700 may include one or more of the following components: a processing component 702, a memory 704, a power component 706, a multimedia component 708, an audio component 710, an input/output (1/0) interface 712, a sensor component 714, and a communication component 716.

The processing component 702 usually controls overall operations of the device 700, such as the operations associated with display, telephone calls, data communications, camera operations, and recording operations. The processing component 702 may include one or more processors 718 to execute instructions to perform all or part of the steps in the above-described methods. Moreover, the processing component 702 may include one or more modules which facilitate the interaction between the processing component 702 and other components. For instance, the processing component 702 may include a multimedia module to facilitate the interaction between the multimedia component 708 and the processing component 702.

The memory 704 is configured to store various types of data to support the operation of the device 700. Examples of such data include instructions for any application or method operated on the device 700, contact data, phonebook data, messages, pictures, videos, etc. The memory 704 may be implemented using any type of volatile or non-volatile memory device or combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The power component 706 provides power to various components of the device 700. The power component 706 may include a power management system, one or more power sources, and other components associated with the generation, management, and distribution of power in the device 700.

The multimedia component 708 includes a screen providing an output interface between the device 700 and the user. In some embodiments, the screen may include a liquid crystal display (LCD) and a touch panel (TP). If the screen includes the touch panel, the screen may be implemented as a touch screen to receive input signals from the user. The touch panel includes one or more touch sensors to sense touches, slips, and gestures on the touch panel. The touch sensors may not only sense a boundary of a touch or slip action, but also sense a period of time and a pressure associated with the touch or slip action. In some embodiments, the multimedia component 708 includes a front camera and/or a rear camera. The front camera and/or the rear camera may receive an external multimedia datum while the device 700 is in an operation manner, such as a photographing manner or a video manner. Each of the front camera and the rear camera may be a fixed optical lens system or have focus and optical zoom capability.

The audio component 710 is configured to output and/or input audio signals. For example, the audio component 710 includes a microphone configured to receive an external audio signal when the device 700 is in an operation manner, such as a call manner, a recording manner, and a voice identification manner. The received audio signal may be further stored in the memory 704 or transmitted via the communication component 716. In some embodiments, the audio component 710 further includes a speaker to output audio signals.

The I/O interface 712 provides an interface between the processing component 702 and peripheral interface modules, such as a keyboard, a click wheel, a button, and the like. The button may include, but not limited to, a home button, a volume button, a starting button, and a locking button.

The sensor component 714 includes one or more sensors to provide status assessments of various aspects of the device 700. For instance, the sensor component 714 may detect an open/closed status of the device 700, relative positioning of components, e.g., the display and the keyboard, of the device 700, a change in position of the device 700 or a component of the device 700, a presence or absence of user contact with the device 700, an orientation or an acceleration/deceleration of the device 700, and a change in temperature of the device 700. The sensor component 714 may include a proximity sensor configured to detect the presence of nearby objects without any physical contact. The sensor component 714 may also include a light sensor, such as a CMOS or CCD image sensor, for use in imaging applications. In some embodiments, the sensor component 714 may also include an accelerometer sensor, a gyroscope sensor, a magnetic sensor, a pressure sensor, or a temperature sensor.

The communication component 716 is configured to facilitate communication, wired or wirelessly, between the device 700 and other devices. The device 700 may access a wireless network based on a communication standard, such as WiFi, 2G, 3G, or a combination thereof. In one exemplary embodiment, the communication component 716 receives a broadcast signal or broadcast associated information from an external broadcast management system via a broadcast channel. In one exemplary embodiment, the communication component 716 further includes a near-field communication (NFC) module to facilitate short-range communications. For example, the NFC module may be implemented based on a radio frequency identification (RFID) technology, an infrared data association (IrDA) technology, an ultra-wideband (UWB) technology, a Bluetooth (BT) technology, and other technologies.

In exemplary embodiments, the device 700 may be implemented with one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing the above-described methods.

In exemplary embodiments, there is also provided a non-transitory computer readable storage medium including instructions, such as included in the memory 704, executable by the processor 718 in the device 700, for performing the above-described methods. For example, the non-transitory computer-readable storage medium may be a ROM, a random access memory (RAM), a CD-ROM, a magnetic tape, a floppy disc, an optical data storage device, and the like.

One of ordinary skill in the art will understand that the above-described modules can each be implemented by hardware, or software, or a combination of hardware and software. One of ordinary skill in the art will also understand that multiple ones of the above-described modules may be combined as one module, and each of the above-described modules may be further divided into a plurality of sub-modules.

Other embodiments of the present disclosure will be apparent to those skilled in the art from consideration of the specification and practice the present disclosure. This application is intended to cover any variations, uses, or adaptations of the present disclosure following the general principles thereof and including such departures from the present disclosure as coming within common knowledge or customary technical means in the art. It is intended that the specification and embodiments be considered as exemplary only, with a true scope and spirit of the present disclosure being indicated by the appended claims.

It should be understood that the present disclosure is not limited to the exact construction that has been described above and illustrated in the accompanying drawings, and may be modified and changed without departing from the scope of the present disclosure. It is intended that the scope of the invention is only defined by the appended claims.

What is claimed is:

1. A method for a device to acquire air quality, comprising:
controlling a fan to rotate;
measuring air quality by an air-quality detector; and
generating air-quality information according to a measurement result of the air-quality detector.

2. The method according to claim 1, wherein the measuring of the air quality by the air-quality detector comprises:
detecting whether a rotation time of the fan reaches a predetermined length of time; and
measuring the air quality through the air-quality detector if the rotation time reaches the predetermined length of time.

3. The method according to claim 1, wherein the controlling of the fan to rotate comprises:
detecting whether an air-flowing speed in an environment surrounding the device reaches a predetermined speed; and
controlling the fan to rotate if the air-flowing speed does not reach the predetermined speed.

4. The method according to claim 1, further comprising:
receiving a trigger signal sent by a mobile terminal, the trigger signal being configured to trigger the measurement of the air-quality; and
controlling the fan to rotate according to the trigger signal.

5. The method according to claim 4, further comprising at least one of:
sending the air-quality information to the mobile terminal for displaying;
sending the air-quality information to a server, the server being configured to send the air-quality information to the mobile terminal for displaying; or sending the air-quality information to a gateway device, the gateway device being configured to send the air-quality information to the mobile terminal for displaying.

6. The method according to claim 1, further comprising:
detecting whether a predetermined time is reached, the predetermined time being one of a plurality of times at a predetermined time interval, or a time set by a user; and
controlling the fan to rotate if the predetermined time is reached.

7. The method according to claim 6, further comprising:
sending the air-quality information to at least one of a server or a gateway device, the at least one of the server or the gateway device being configured to:
receive from a mobile terminal a trigger signal for displaying air-quality information; and
send to the mobile terminal most recently received air-quality information or all historically received air-quality information.

8. A device for acquiring air quality, comprising:
a processor; and
a memory for storing instructions executable by the processor;
wherein the processor is configured to perform:
controlling a fan to rotate;
controlling an air-quality detector to measure air quality; and
generating air-quality information according to a measurement result of the air quality detector.

9. The device according to claim 8, wherein the processor is further configured to perform:
detecting whether a rotation time of the fan reaches a predetermined length of time; and
controlling the air quality detector to measure the air quality if the rotation time reaches the predetermined length of time.

10. The device according to claim 8, wherein the processor is further configured to perform:
detecting whether an air-flowing speed in an environment surrounding the device reaches a predetermined speed; and
controlling the fan to rotate if the air-flowing speed does not reach the predetermined speed.

11. The device according to claim 8, wherein the processor is further configured to perform:
receiving a trigger signal sent by a mobile terminal, the trigger signal being configured to trigger the measurement of the air quality; and
controlling the fan to rotate according to the trigger signal.

12. The device according to claim 11, wherein the processor is further configured to perform at least one of:
sending the air-quality information to the mobile terminal for displaying;
sending the air-quality information to a server, the server being configured to send the air-quality information to the mobile terminal for displaying; and
sending the air-quality information to a gateway device, the gateway device being configured to send the air-quality information to the mobile terminal for displaying.

13. The device according to claim 8, wherein the processor is further configured to perform:
detecting whether a predetermined time is reached, the predetermined time being one of a plurality of times at a predetermined time interval, or a time set by a user; and
controlling the fan to rotate if the predetermined time is reached.

14. The device according to claim 13, wherein the processor is further configured to perform:
sending the air-quality information to at least one of a server or a gateway device, the at least one of the server or the gateway device being configured to:
receive from a mobile terminal a trigger signal for displaying air-quality information; and
send to the mobile terminal most recently received air-quality information or all historically received air-quality information.

15. A non-transitory computer-readable storage medium storing instructions that, when executed by a processor of a device, cause the device to perform a method for acquiring air quality, the method comprising:
controlling a fan to rotate;
measuring air quality by an air-quality detector; and
generating air-quality information according to a measurement result of the air quality detector.

16. The medium according to claim 15, wherein the measuring of the air quality by the air-quality detector comprises:
detecting whether a rotation time of the fan reaches a predetermined length of time; and
measuring the air quality through the air-quality detector if the rotation time reaches the predetermined length of time.

17. The medium according to claim 15, wherein the controlling of the fan to rotate comprises:
detecting whether an air-flowing speed in an environment surrounding the device reaches a predetermined speed; and
controlling the fan to rotate if the air-flowing speed does not reach the predetermined speed.

18. The medium according to claim 15, wherein the method further comprises:
receiving a trigger signal sent by a mobile terminal, the trigger signal being configured to trigger the measurement of the air-quality; and
controlling the fan to rotate according to the trigger signal.

19. The medium according to claim 18, wherein the method further comprises at least one of:
sending the air-quality information to the mobile terminal for displaying;
sending the air-quality information to a server, the server being configured to send the air-quality information to the mobile terminal for displaying; and
sending the air-quality information to a gateway device, the gateway device being configured to send the air-quality information to the mobile terminal for displaying.

20. The medium according to claim 15, wherein the method further comprises:
detecting whether a predetermined time is reached, the predetermined time being one of a plurality of times at a predetermined time interval, or a time set by a user; and
controlling the fan to rotate if the predetermined time is reached.
